(19) United States
(12) Patent Application Publication
(10) Pub. No.: US 2003/0223905 A1
(43) Pub. Date: Dec. 4, 2003

(54) Method and apparatus for quantifying caloric balance using metabolic parameters to assist subjects on weight management

(76) Inventor: Piet Moerman, St. Martens-Latem (BE)

Correspondence Address: LAHIVE & COCKFIELD, 28 STATE STREET, BOSTON, MA, 02109, US

(21) Appl. No.: 10/400,345

(22) Filed: Mar. 26, 2003

Related U.S. Application Data
(60) Provisional application No. 60/367,808, filed on Mar. 26, 2002.

(51) Int. Cl. G01N 33/00

(52) U.S. Cl. 422/56; 436/60; 436/128

(57) ABSTRACT

A weight loss system and method measures one or more metabolic parameters in a body fluid sample and correlates the level of the metabolic parameter to a change in body fat or a metabolic state. The weight loss system includes a weight loss monitor including a sampling device for yielding a sample of the body fluid sample and a test element for analyzing the body fluid sample to determine the level of the metabolic parameter. An algorithm correlates the level of the metabolic parameter to a change in body fat, the metabolic state of the user or other parameter indicative of the success of the dieting process. The weight loss monitor may also track and display a user's weight versus an objective and provide feedback and assistance with the dieting process.

### RELATED APPLICATIONS

The present application claims priority to U.S. Provisional Patent Application Serial No. 60/367,808, filed Mar. 26, 2002, and entitled “A Method and Apparatus for Quantifying Caloric Balance Using Metabolic Parameters to Assist Subjects on Weight Management”, the contents of which are herein incorporated by reference.

### FIELD OF THE INVENTION

The present invention relates to a system and a method for managing body weight using the quantification of biochemical markers in the subject to assess an actual fat burning state and to quantify the amount of body fat consumed.

### BACKGROUND OF THE INVENTION

The prevalence of obesity is increasing at an alarming rate. The percentage of Americans considered overweight has soared from 12% in 1991 to 18% in 1998. As many as one in three adults in the United States are overweight, or roughly 58 million people. In particular, the rise in adolescent obesity is causing great concern. Obesity is becoming one of the main risk factors in the development of the so-called “western” diseases. Americans spend almost $70 billion on health complications linked to being overweight. Another $33 billion a year is spend on weight loss products and programs.

A series of diseases are directly associated with being overweight. For example, 83% of all diabetes patients are overweight. Between 1990 and 1998, the prevalence of diabetes in the United States rose from 4.9 to 6.5%. During the 1990's, the prevalence of type 2 diabetes increased by 33% overall and by 70% among people in their thirties. Diabetes now affects 16 million Americans. The direct costs affecting diabetes is $44 billion and including all indirect costs $98 billion. 13.5% of obese patients have diabetes compared to 3.5% of those with a normal weight. Weight control Intervention is desired and proven to be the number one step in the therapy of most Type 2 diabetics.

Cardiovascular Disease is also related to obesity. Killing almost a million Americans per year, cardiovascular disease—principally hypertension, heart disease, and stroke—is the leading cause of death among both men and women, and across all racial and ethnic groups. About 58 million Americans live with some form of the disease. In 1999 alone, cardiovascular disease cost the nation an estimated $287 billion and this burden is growing as the population ages. A limited number of health related behaviors—most notably tobacco use, lack of physical activity, and poor nutrition—are responsible for much of the burden. The major classes of cardiovascular disease such as Hypertension, Arteriosclerosis, Acute Myocardial Infarction and Chronic Heart Failure have a weight component in the pathogenesis or therapeutic intervention. Even a modest reduction on weight (5-10%) can reduce the risk of serious health problems.

Weight control programs are primarily behavior modification programs aiming to changing the behavior in calorie consumption and possibly also exercise. According to the behavioristic psychology a couple of tools are essential or helpful in the success of changing behavior: setting objective and attainable goals, short term feedback and incentives, reporting the progress towards the set goal, sustainability of the required effort over the entire course until the goal is attained. So far, no method has been in place to allow short-term feedback based on an objective parameter. Most weight control programs concentrate on weight as the monitoring parameter. Within the current thinking of the behavioristic psychology, one needs a faster reactive and more objective parameter to motivate patients to sustain their efforts.

### SUMMARY OF THE INVENTION

The present invention provides for an electronic weight loss monitoring device employing a weight management program that measures and utilizes metabolic analytes, such as ketones, glycerol or free fatty acids (FFA), as objective monitoring tools for the dieting process. The metabolic analytes are a side product of body fat breakdown and hence increase when a patient restricts their caloric intake. These objectively measurable parameters are used to assess the caloric deficit of the patient or the amount of fat lost by the patient when restricting their caloric intake. An analysis of the levels of one or more of the parameters, measured at specific times during the day, reflects the amount of body fat the body has utilized. Since these levels are objectively measurable parameters, they eliminate the subjective and often erroneous traditional methods of counting calories used in current weight management programs. In addition, an analysis of metabolic analyte levels reflects the actual amount of fat burned rather than an indirect assumption of the amount based on calorie intake counting.

The weight loss monitoring device may further use other biological or other parameters, such as sex, weight, body mass index (BMI) or body composition, duration of caloric restriction (diet), exercise intensity and duration and meal composition, to calculate lipolysis (i.e. loss of body fat) for the user. The program and device of the present invention may further calculate or determine the appropriate caloric deficit incurred or an amount of fat burned by the user at any particular moment, and translate or convey this data into the amount of calories the subject can consume while maintaining compliance with an established weight loss goal.

According to one aspect, an electronic weight loss monitoring device of the present invention includes a hand-held ketone sensor or fluid sampling device that measures ketone levels in a biological fluid, such as blood, using a test strip and a skin lancing device to produce a fluid sample. The electronic weight loss monitoring device uses one or more parameters, such as sex, weight, body mass index (BMI), duration of caloric restriction (diet), exercise intensity, exercise duration and meal composition, and measures a metabolic analyte, such as the total KB or FFA or glycerol concentration to calculate lipolysis (i.e. loss of body fat for the user). The monitoring device may also measure analyte levels and inform a user regarding his actual metabolic state (e.g., anabolic or catabolic). The electronic device may also compare the output to a set objective for the user and tracks the output against the set objective on a screen. The electronic device may further calculate a calorie deficit in the user and an allowed caloric intake for the next meal. The device may output the calculated allowed caloric intake to the user. The electronic device may further suggest a meal composition that complies with the calculated allowed caloric intake.

The device and method of the present invention provide short feedback to the patient, within 24-36 hours after initial calorie restriction, and 4-6 hours while on a diet, due to the rapid response of FFA, ketones and glycerol. The present invention provides short-term incentives as well. By providing a short loop feedback of the actual fat metabolic state, the patient may be more motivated to maintain a dieting program, even when his efforts are not translated yet into a noticeable reduction of his weight.

According to an alternate embodiment, the present invention can be used to help people gain weight, by avoiding the generation of those metabolites from fat breakdown.

The present invention offers tools for setting realistic objectives, tracking results, short loop feedback and motivating consumers based on these objective metabolic signals. The invention further enhances the degrees of freedom and flexibility when on a weight loss regimen or a weight maintenance program.

The present invention also facilitates assessment of body composition. To assess body composition, baseline FFA are correlated for the proportion of body fat in a subject. Baseline values of the analytes can therefore be used for tracking long term improvement in body composition, an essential end point in the treatment of pre-diabetes.

According to one aspect of the invention, a method is provided comprising the steps of measuring a metabolic analyte in a biological fluid of a user, and correlating the metabolic analyte to one of an amount of body fat of the user and a change in the amount of body fat of the user. The method may include the step of correlating further comprises the step of correlating an amount of body fat lost by the user to an expected change in body weight of the user. The method may further include the step of calculating a calorie deficit incurred by the user. In one aspect, the method further comprises the step of displaying a selection of meals having the amount of calories calculated in said step of calculating.

According to another aspect of the invention, a device is provided, which comprises measurement means for measuring a metabolic analyte in a biological fluid of a user, and a processor for correlating the metabolic analyte to a change in an amount of body fat in the user.

According to another aspect of the invention, an apparatus for measuring and correlating a metabolic analyte in a sample to one of weight loss and body fat loss is provided. The apparatus comprises a sampling device for receiving a biological fluid from a user, the biological fluid including a metabolic analyte, a test element for measuring an amount of the metabolic analyte in the biological fluid, and a processor for calculating the amount of the metabolic analyte in the biological fluid, and for correlating the metabolic analyte to one of an amount of body fat of the user and a change in the amount of body fat of the user.

In yet another aspect, a method comprising the steps of measuring a metabolic analyte in a biological fluid of a user, and correlating the metabolic analyte to a biological parameter of the user is provided.

### BRIEF DESCRIPTION OF THE DRAWINGS

FIGS. 1a and 1b illustrate a personal weight loss monitoring device according to one embodiment of the present invention.

FIGS. 2a and 2b illustrate embodiments of a test element employed by the weight loss monitoring device of the present invention.

FIG. 3a is a schematic of a health monitoring system including the health monitoring device of FIGS. 1a and 1b.

FIG. 3b is a block diagram showing the components of the processor of FIG. 3a.

FIG. 4 is an illustration of the display of the monitoring device when comparing an actual weight to an objective weight.

FIG. 5 is an illustration of the display of the monitoring device when measuring ketone levels.

FIG. 6 is an illustration of the monitoring device when using the monitor as a meal assistant.

FIG. 7 illustrates an embodiment of the monitoring device when the sampling and testing functions are integrated into a single device.

### DETAILED DESCRIPTION OF THE INVENTION

The present invention provides a method, device and program for efficiently, effectively, easily and safely monitoring weight loss in a patient. The invention will be described below relative to an illustrative embodiment. Those skilled in the art will appreciate that the present invention may be implemented in a number of different applications and embodiments and is not specifically limited in its application to the particular embodiments depicted herein.

As used herein, the term “metabolic analyte” refers to an analyte generated in a patient when consuming body fat. Metabolic analytes include, but are not limited to, ketones, free fatty acids (FFA), and glycerol.

As used herein, the term “lipolysis” refers to fat breakdown in the body.

The term “biological fluid” as used herein refers to a fluid containing a metabolic analyte, including, but not limited to blood, derivatives of bloods, interstitial fluid, urine, a breathe sample, saliva, and combinations thereof.

As used herein, the term “biological parameter” is intended to include any parameter associated with the biology of the user, examples of include an amount of body fat in the user, a change in the amount of body fat in the user, a metabolic rate of the user, weight, caloric consumption, calorie burning rate.

As used herein, the term “network” is intended to include any suitable arrangement of electronic devices, examples of which include an Internet, an extranet, an intranet, a wide area network (WAN), a metropolitan area network (MAN), a local area network (LAN), a satellite network, a wireless network, or some other type of network.

The present invention provides a system and method for monitoring weight loss by utilizing one or more parameters, such as a metabolic parameter, including a metabolic analyte, and optionally one or more other parameters, such as sex, weight, body mass index (BMI) or body composition, duration of caloric restriction (diet), exercise intensity, exercise duration, and meal composition.

A weight loss monitoring device of one embodiment of the present invention correlates one or more metabolic analytes and optionally other parameters of a dieting subject with a biological parameter of the user, such as the actual fat loss in the dieting subject. Suitable metabolic analytes, as set forth above, include ketones, such as beta-Hydroxybutyrate (one of the three types of ketones in the body), free fatty acids which are released from the body fat tissue, and glycerol which appears in circulation when free fatty acids are released from the body fat stores. Normally, levels of beta-Hydroxybutyrate are expected to be less than 0.6 mmol/l and these levels increase if a person fasts or exercises vigorously.

The present invention will be described relative to an embodiment wherein a metabolic analyte, such as a level of ketones, is measured and correlated to a metabolic consumption rate, a change in body fat, an amount of body fat, a caloric consumption rate or other biological parameter. Those of ordinary skill will readily recognize that other metabolic analytes can also be used. Ketone formation resulting from fat utilization increases with increasing energy demand. Typically, ketone levels increase as an individual burns more energy while exercising or otherwise being physically active. Therefore, ketone levels can be used to monitor weight since it integrates the extra calories lost when combining diet with an exercise plan. Other body fat metabolites follow a similar pattern in function of fat utilization as an energy source and can be used for the same purposes. However, reaction time and analyte levels differ significantly from ketone levels.

According to an illustrative embodiment, ketone levels in a biological fluid, such as blood, may be measured using any conventional fluid sampling device, such as the commercially available device sold under the tradename Medisense Precision Extra, by Medisense, USA. One skilled in the art will readily recognize that any suitable sampling device for measuring a metabolic analyte, such as ketone levels, and additional metabolic analytes may be employed, including devices that analyze a patient's breathe, urine or saliva to determine ketone levels.

The device of the illustrative embodiment, described in detail below, utilizes ketone strips to measure ketone levels in a blood sample, in a manner similar to the process of measuring glucose levels in a blood sample, though one skilled in the art will recognize that other means for measuring a metabolic analyte may be used.

During caloric intake restriction (i.e., dieting, starvation and the like), the human metabolism changes in order to access existing energy reserves. In the first 1-2 days after initiating starvation, glucose concentration in the blood is typically maintained primarily through utilizing the glucose storage (glycogen) in the liver and muscle. After about 36 hours of starvation, newly formed glucose (gluconeogenesis) provides 75% of the liver production of glucose. About 50% of this glucose is derived from muscle protein breakdown, the remainder from fat breakdown. After about 5-7 days of caloric intake restriction, protein consumption for energy delivery is almost entirely replaced by fat consumption. This body fat consumption is usually the goal of therapeutic starvation or dieting.

Even within the period of an overnight fast, the drop in plasma insulin is sufficient to cause significant lipolysis with the release of fat metabolites, such as free fatty acids, glycerol and ketones, into the blood. The liver responds to the resulting rise in glucagon/insulin ratio by increasing the beta-oxidation of the free fatty acids leading to the formation of ketones. The rate of this ketogenesis continues to increase as caloric restriction is maintained.

Blood ketone concentrations typically change more dramatically than FFA concentrations, showing a more than 50-fold increase (to 0.6 mM) in the first two days of starvation, whereas FFA levels tend to rise between 2 and 3 fold. Ketones are an excellent fuel source for a wide variety of body tissues, and their production by the liver can be regarded as producing extra energy during the consumption of Free Fatty Acids. Glycerol mainly ends up in the gluconeogenesis, producing glucose for tissue and blood cells.

In diabetic subjects, where the glucagon/insulin ratio can be very high, ketone levels can surge to 6 mmol/l. These unusually high levels are not seen in normal healthy individuals and are problematic for the individual. Such high levels among diabetics are the result of lack of intake, rather than dietary restrictions.

The weight loss monitoring device of the present invention utilizes a program that converts a measured concentration of one or more metabolic analytes into usable and easily readable information. For example, ketone levels can be measured. Ketone levels above a certain value indicate that the user is actually in a catabolic state, while ketone levels below a certain value indicate that a user is in an anabolic state.

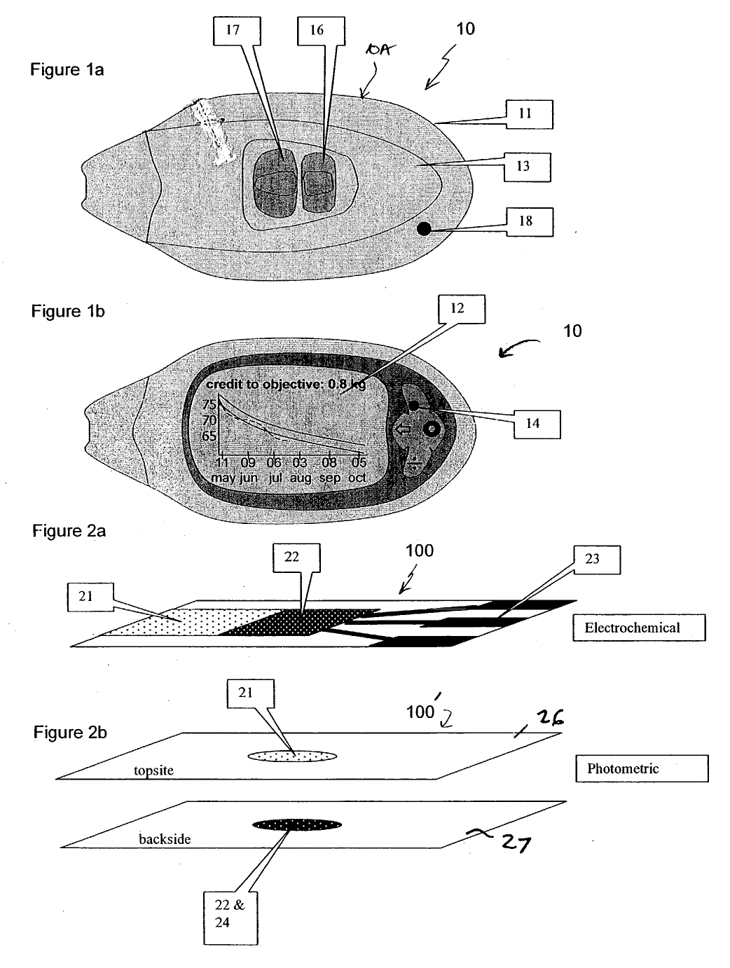

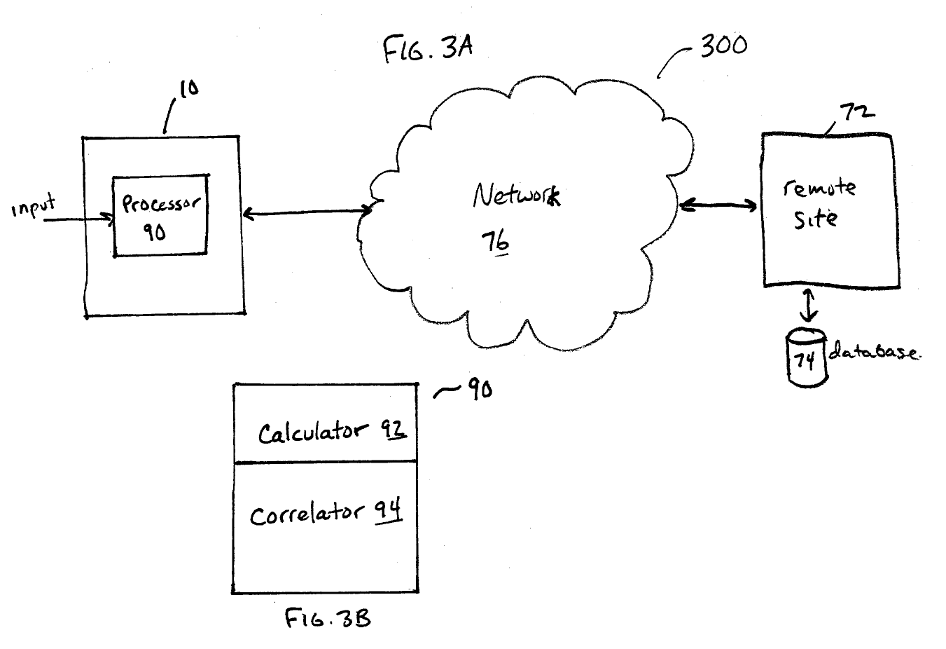

According to an illustrative embodiment of the present invention, the weight loss monitoring system of the present invention includes a personal weight monitor 10, shown in FIGS. 1a and 1b, comprising a sampling device 10A and a test element 100 (FIG. 2a). The sampling device 10A samples and measures a parameter, such as the presence, absence or amount of a metabolic analyte, related to fat metabolism in a biological fluid. The test element 100 is used to quantify the analyte. The test element 100 may be disposable or non-disposable. In one embodiment, the weight loss monitoring system of the present invention utilizes a processor 90, shown in FIGS. 3a and 3b, configured and adapted to determine an appropriate frequency of testing and for calculating a biological parameter, such as the amount of body fat utilized, or burned, based on the level of the measured metabolic analyte in the user, obtained using the sampling device 10A. The measurement functionality of the device 10 can be performed or integrated in one or more of the test strip, the processor or one or more other components of the device.

The personal weight monitor 10 may further include a weight management program and interface that allows a user to set objectives, and provides a feedback mechanism on the user's actual performance versus one or more set objectives, as well as behavioristic tools to motivate the user. The system may further include a dietary database stored in the sampling device from which the user can select meal compositions within the proposed caloric restriction. The system may also be configured to remotely communicate with the database through a network to facilitate interfacing, program selection, customizing the apparatus software and data downloading.

The illustrated personal weight monitor 10 shown in FIGS. 1a and 1b measures a metabolic analyte in a user. The personal weight monitor 10 correlates or converts the measured analyte level into a metabolic consumption rate, or other biological parameter, and informs the user regarding his or her actual metabolic fat consumption state, either anabolic or catabolic. The personal weight monitor 10 provides a relatively short feed back opportunity for the user (i.e., 24-36 hours after initial calorie restriction and 4-6 hours while on diet). The user may conclude, upon positive test results, that body fat is being consumed even if a change in body weight is not yet noticeable.

With reference to FIG. 1a, the personal weight monitor 10 comprises a housing 11 encompassing a display 12, as well as other electrical, mechanical, and/or chemical components, as would be obvious to one of ordinary skill, including but not limited to PCB, storage elements including programmed storage elements, a test element connector, a battery compartment 13, and menu navigation buttons 14, shown in FIG. 1b.

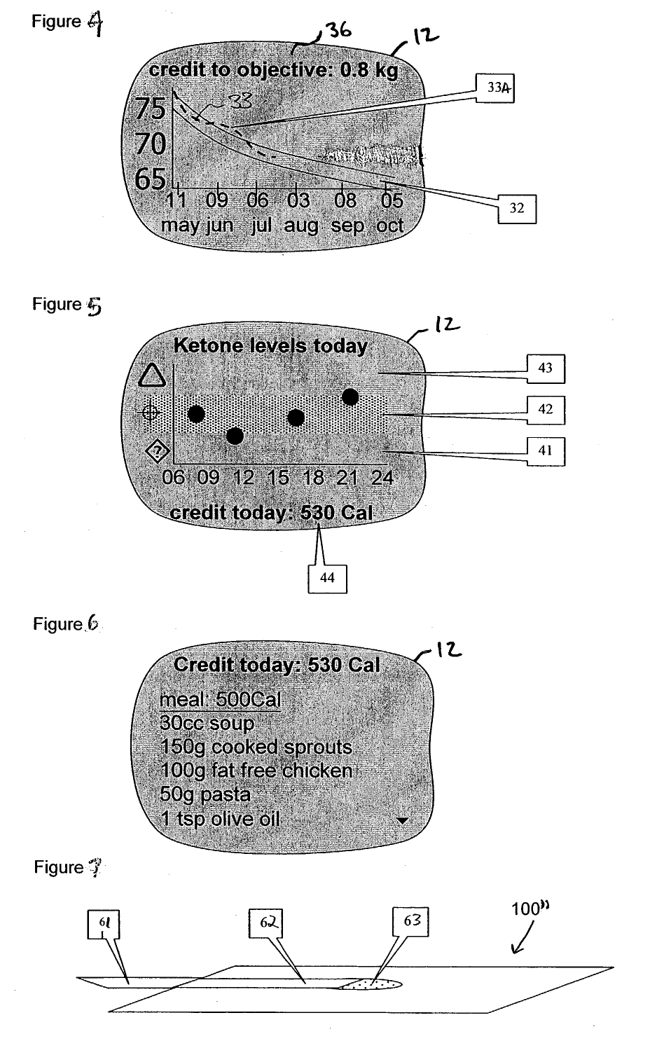

As illustrated in FIG. 1b and in FIG. 4, the display 12 of the personal weight monitor 10 may be used to track the user's weight over a period of time and display an objective weight. The monitor 10 compares the user's actual weight against an objective curve to provide feedback to the user regarding his progress.

According to one practice, a preferred frequency of testing of the metabolic analyte may be determined by the degree of caloric restriction. For example, for a low calorie deficit regimen, the personal weight monitor 10 may test metabolic analytes in the afternoon, for example, around 4:00 p.m., and at least 3 hours past lunchtime. For a moderate calorie deficit regimen, the personal weight monitor 10 preferably tests metabolic analytes at pre-lunch and pre-dinner moments. For a high calorie deficit regiment, the monitor preferably tests metabolic analytes before all three meals.

From certain levels on, the weight loss monitoring system of the invention may guide the user to test more frequently, i.e. if the afternoon level is high enough, one can expect the pre-lunch values to start becoming positive as well. The system may also guide the user to test his metabolic analytes during and after exercise, i.e., for example post exercise ketones.

According to one embodiment, an exercise program may be used to supplement and assist the system in monitoring weight loss. Such an algorithm takes into account faster burn rates when combined with an exercise program, as well as the peculiar effects of exercise on the tested analyte levels.

Referring again to FIGS. 1a through 2b, the illustrated personal weight monitor 10 includes a sampling device 10A for yielding a selected volume of biological fluid, such as blood, to be tested for a selected parameter, such as a metabolic analyte. Suitable sampling devices include known lancing devices and other sampling devices for yielding a biological fluid for testing, such as for blood glucose testing. In the illustrative embodiment, the sampling technology for measuring ketone levels may be similar to the “stick and read” ICF method of Integ, USA, though one of ordinary skill in the art will recognize that the invention is not limited to such a sampling device. The advantages to such testing are that the testing is painless, fast, technique independent and does not require large quantities of blood. These lancing devices are spring-loaded devices which cause a lancet to penetrate the skin. Other devices have been described to yield interstitial fluid (Integ) and are suitable for use in the present invention. Both methods, yielding blood and interstitial fluid, can be used with the weight loss monitoring system and method of the present invention for obtaining a sample of body fluid in order to measure a metabolic analyte in a user.

Those of ordinary skill will readily recognize that the sampling device may be a separate stand alone device, or may be incorporated in the personal weight monitor 10, as illustrated by the sampling device 10A. The sampling device and the test element can be constructed in such a way to achieve an automatic sample transfer from the place of yielding the sample to the test element. As shown in FIG. 1a, the sampling device may include a cocking button 16 for loading a piercing element, such as a lancet, and a variable penetration depth button 17 for setting the penetration depth of the lancet. Suitable depth adjustment mechanisms are known in the art, and need not be described in detail herein.

According to another embodiment, ketone levels and other metabolic analytes may be measured using a breathalyzer or by analyzing urine samples, saliva samples, or other biological fluid samples.

The test element 100 of the personal weight monitor 10 quantifies the amount of one or more metabolic analytes, such as ketones, glycerol or free fatty acids, in a sample obtained by the sampling device, which are generated in a user when consuming body fat. The test element 100 generates a signal indicative of the concentration of the tested metabolic analyte in the sample, which can be based either on a photometric or electrochemical analytical method. Various ketone test methods currently sold in the marketplace include a photometric test method by Gupta Diagnostic Systems under the tradename Ketosite, and by Polymer Technology Inc under the tradename BioScanner 2000. Electrochemical test elements are sold by MediSense, such as under the tradename Precision Extra. Various tests for glycerol and FFA are also readily available and are known to those of ordinary skill in the art.

As shown in FIGS. 2a and 2b, the illustrated test element 100 consists typically of a sample application zone 21 and an analysis portion 22. In case of an electrochemical test, as shown in FIG. 2a, the test element 100 features an electrical contact zone 23 that may include a plurality of electrodes to make contact through a strip connector mounted in the housing 11 with any electronics disposed within the sampling device 10. Test elements of this type are known in the art and need not be described further herein.

The personal weight monitor 10 of the present invention may also be configured to employ a different type of test strip 100′. For example, as shown in FIG. 2b, a photometric strip 100′ can be used. The strip includes a sample application zone 21 disposed on a first layer 26, and an analysis zone 22 and a detection zone 24 disposed on a second layer 27. The application zone 21 on the first layer 26 can be in fluid or optical communication with zones 22 and 24 on the second layer 27. In operation, a color change that occurs in one of the layers is measured by means of reflecto-photometry rather than an electrical current. Test strips of this type are known in the art and need not be described further herein.

The illustrated test elements 100 and 100′ may feature a built in sampling device 10A that eliminates a manual transfer of the biological fluid sample from the sampling device to the application zone of the test element. For example, according to one embodiment, as shown in FIG. 7, the skin penetration member 61 in the sampling device is part of the disposable test device 100″ and works in conjunction with the personal weight monitor 10. The illustrated skin penetration member 28 pierces the skin to yield a drop of body fluid. The test element also includes a sample channel 62 for conveying the body fluid through the test element 100″ to a detection zone 63. The detection zone analyzes the body fluid to determine or measure an amount of a metabolic analyte in the body fluid.

The test elements 100, 100′, and 100″ may be individually loaded into the personal weight monitor 10 by the user, or can be pre-packaged in a cassette that contains multiple test elements for easier loading.

The illustrated personal weight monitor 10 contains electronics, including a processor 90 for reading and receiving a signal from the test elements, shown in FIGS. 3a and 3b. By using the calibration information for the test element, the processor can convert the measured signal generated by the test element to a concentration of the tested metabolic analyte. The processor 90 provides feedback to a user based on a level of a metabolic analyte in a biological fluid sample. The processor 90 includes a calculator 92 for determining the level of the metabolic analyte in the sample and a correlator 94 for correlating the level of the metabolic analyte to a biological parameter indicative of weight loss or gain. The measured analyte concentration can be displayed on the display 12 and/or stored into memory of the monitor 10. The processor 90 may include any combination of a caloric determination program for calculating a calorie deficit incurred by the user, a meal assistant program for providing a list of suitable meals for the user, a weight loss program for calculating an amount of weight lost, a metabolism program for calculating the user's metabolism rate, an exercise program for calculating the effect of exercise on the body and a behavior determination program for measuring and providing feedback to the user regarding his progress. For example, the stored readings may be used by a program module to calculate an amount of fat or calories consumed by the user based on the metabolic analyte levels and/or to calculate an amount of calories the user may consume while maintaining compliance with his established weight loss objective. The monitor 10 may then inform the user regarding the amount of fat or calories he may have lost or the amount of calories he is allowed to eat within compliance to his set weight loss objective.

In one embodiment, as shown in FIG. 3a, the monitor 10 may form part of a weight loss monitoring system 300. The weight loss monitoring system comprises the monitor 10 and a remote site 72 having a database 74 for storing data obtained by the monitor 10. As shown, the monitor may be connected to the remote site 72 over a network 76.

The weight loss monitoring system of the present invention may include suitable code and hardware for interfacing with the network 76 (e.g., a web browser), program selection, software customization, data downloading, and the like. Through the network 76, the user can change the settings of his apparatus as described below. The more convenient interface provides enhanced ease of use. The settings may then be loaded from the website through the PC into the personal weight monitor via the communication port 18. The network may further allow the personal weight monitor 10 to access education and behavior modification programs to inform the user regarding dieting.

According to one embodiment, the personal weight monitor 10 through the network 76 may download and store certain programs, such as dieting programs for specific situations. For example, the user may select a sports diet program providing sufficient carbohydrates for energy supply and more proteins for muscle mass building. A Type 2 diabetic can select a Type 2 Diabetes program that concentrates on the interaction of the taken medication in concert with the weight loss program. A reconvalesense program can help the user to regain both fat and protein mass. A weight maintenance program will aim at maintaining the subject's weight while allowing for more freedom in caloric intake. The personal weight monitor 10 can either store the users information locally (i.e., within the device) or can in a database at a remote site through the network. The user data can be stored at the remote site 72 to form a personalized database 74 or record. Pattern recognition software may be employed at any point of the system to recognize typical trends in order to identify problems, such as yo-yo dieting, aggressive weight expectations, weekend deviations and the like, while concomitantly offering (if desired) a tailor-made education package that is customized to the user.

The weight loss monitoring monitor of the present invention may further include a behavior modification interface for goal setting and motivation. Any behavior modification program, such as a diet to lose or gain weight, is driven by the setting of realistic and attainable objectives. The system of the present invention may assist a user in setting his weight loss objectives. The user may input or enter his current weight, sex and height into the personal weight monitor 10. The system then calculates an ideal weight for the user based on the entered parameters, which the user can accept or change to a different value. The system allows the user to select the intensity of the dieting program (mild, intensive, very intensive). The level of intensity determines the time span over which the user should attain his desired weight.

As shown in the display 12 of FIG. 4, the monitor 10 logs the entered weight of the user, graphs the user's weight, shown by line 33, against time, and compares the user's actual weight, line 33, to the objective weight 32. Rather instructing the user to lose a fixed amount of weight (e.g. ×kg/d), the monitor 10 calculates the time span based upon a more realistic percentage weight loss per day to achieve the objective. This results in an asymptotic-like objective curve 31 showing the users weight goal over time. The objective curve 32 may also show middle-term objectives. As weight can vary because of physiological events such as a woman's menstrual cycle, degree of hydration, salt intake, recent urine void, and the like, the middle-term objectives are displayed as a “zone” 32 rather than a rigid number. In this manner, representing the individual's performance is more forgiving and may allow for some deviation of the dieting effort without demotivating the user too much. The user can, while staying within his “goal zone”, put aside the diet regimen (i.e. for a gastronomic weekend, a wedding party or other occasion), as illustrated by the bump 33a in the weight curve 33. The monitor 10 may also calculate and display the users' “credit to objective” amount, as shown in region of the display 12, i.e., the amount of weight he has lost beyond his objective. The freedom and flexibility offered by the monitor 10 of the present invention is an essential part of sustainability over the long-term of dieting programs.

While weight is the long-term compliance marker, the levels of metabolic analytes in the users' system may serve as short-term monitoring markers in the present invention. Metabolic analyte levels may be displayed on demand on the display 12 of the personal weight monitor 10 to show an actual metabolic state (anabolic or catabolic). For example, as shown in FIG. 5, a display 12 shows in any suitable form the level of the analytes in the user over the course of a day. The display may indicate an analyte target level 42, an under-performing range 41, and/or an over-performing range 43. It is useful for the user to be aware of analyte levels in the short terms, as under-performing values do not lead to the desired weight loss, while over-performing values may not be sustainable in the long-term and hold a certain health risk due to over consumption of body proteins.

An integration of these values through the algorithm of the present invention translates the analyte level value to a caloric balance 44. Since the system knows how many calories can be consumed to attain the set goal, the monitor can calculate and display the caloric contents of the meal the user is entitled to. The user can, when performing for awhile at the upper limit, save some “calorie credits” for the next day or weekend. Once again, this flexibility makes part of the behavioristic underpinnings of this method.

As shown in FIG. 6, the weight loss monitoring system of the present invention provides a hassle-free meal assistant that helps the user to predict the allowed caloric intake for the next meal while complying with the set weight loss objective. The system assists the user in planning meals and eliminates calorie intake counting by the user by automatically calculating a calorie balance for the user. The ketone and weight markers will verify the calorie balance. The built up calorie credit may be used to determine future intake. The allowed caloric intake may be coupled to a dietarian database that can suggest menu and meal composition options that comply with the allowed calorie intake.

The form of calorie intake is gained from querying the meal-snack database in the monitor. When the calorie deficit is, for example, 530 Cal, as shown in FIG. 6, the system can provide a selection of meals or snacks not exceeding 530 Cal.

The user can set food and snack preferences during the initial set up of the system. A set up menu can guide the user through a couple of key questions for the selection. For example, the user can enter preferences or dislikes for certain foods, vegetarian habits, allergy restrictions (e.g., gluten free, Yolk free, etc.). During this process, the system assists in the selection of healthy composites. This selection procedure can be greatly facilitated by allowing the personal weight monitor 10 to communicate over the network with a remote location. The monitor may be hooked up to the user's PC through the communication port 18 to download the selected meal-snack database.

According to one embodiment, the personal weight monitor 10 may be capable of communicating with a remote counseling service for providing counseling to the user. The system can then send collected data to a remote location of the counseling service over any communication line, such as a standard phone line, via a PC, or through other suitable means. A communication port 18, shown in FIG. 1a, on the monitor may be used for the data download as well as for receiving information from the website.

More extended information on meal preparation and choices can be made available to the user and the personal weight monitor 10 over the network. The customer can personalize or customize the personal weight monitor 10 by including, for example, specific food requirements. These can include gluten-free diets, calcium rich diets, diets for lactose intolerant subjects, a diet for Phenylketonuria patients, or vegetarian diets.

According to one aspect of the invention, the personal weight monitor 10 can be a personal exercise monitor that displays the amount of calories from fat burnt over an exercise session. The personal exercise monitor tracks and integrates multiple exercise sessions to score against an exercise objective for a user. The personal exercise monitor tracks and integrates multiple exercise sessions to score against an exercise objective for a user.

According to another embodiment, the present invention can be used to help people gain weight to reach a target weight, by tracking and minimizing the generation of those metabolites that results from fat breakdown.

According to another embodiment, the invention is used to simply assess body fat or body composition by measuring baseline values of the analytes. For example, early morning or post-prandial FFA levels in non-dieting subjects correlate with the amount of body fat. This relationship may be utilized to calculate body fat or measure a subject's body composition.

The present invention provides significant advantages over prior systems and methods of monitoring and achieving weight loss in a patient. The system and method of the present invention provide a short feed back opportunity (24-36 hours after initial calorie restriction and 4-6 hours while on diet) to the user on diet. Rather than having to wait several days to see his efforts translated into a weight loss, the present invention provides this information to the user within hours. This short loop feedback allows for short-term incentives.

Further, integration of ketone values tested at certain times during the day correlate with the total amount of fat burned. Therefore, ketone levels translate to the build up caloric deficit. These deficit assessments allow the system to calculate how much the user can eat on his next meal while maintaining his compliance to his set weight objective. The user will be informed how many calories he can take for his next meal.

As the ketone levels give feedback regarding the progress of a diet or weight management program in a matter of only hours, they can be used to provide incentive to the user in the short term. For example, the user may give himself a food treat within the set objective when ketone levels are within a certain range.

Further, certain metabolite levels correlate to the amount of body fat. Therefore, baseline levels of those analytes may be customized to the subject's body composition. Body composition is an important factor in the interpretation of the dieting effect on the measured analytes.

In addition, the use of objectively measurable parameters eliminates the subjective and often erroneous calorie counting in current weight management programs. In addition, an analysis of metabolic analyte levels, in accordance with the teachings of the invention, reflect the actual amount of fat burned rather than an indirect assumption of the amount based on calorie intake counting.

The present invention additionally addresses the medical implications of weight gain and loss and the efficiency of the weight loss and safety of the patient during weight loss. The device and method maintain calorie restriction in the “safe zone” to optimize weight loss while keeping the patient safe (i.e., controlled muscle breakdown) and take into account faster bum rates when combined with an exercise program. The present invention increases short-term success and long term weight maintenance within medically regarded safe limits. The method and device further address the medical implications of weight gain and loss and monitor the efficiency and safety of the weight loss (i.e. muscle breakdown, notably the heart) during the dieting period. The invention further provides short loop feedback of the actual metabolic state for enhanced compliance and assesses the utilized fat for energy in rest as well as the effect of exercise on the dieting subject. Individuals can be oriented to the optimal levels of the fat metabolites (rate of body fat loss) to restrict the caloric intake at the optimal level so to lose a maximum amount of fat in a safe and long term sustainable way.

The present invention has been described relative to an illustrative embodiment. Since certain changes may be made in the above constructions without departing from the scope of the invention, it is intended that all matter contained in the above description or shown in the accompanying drawings be interpreted as illustrative and not in a limiting sense.

It is also to be understood that the following claims are to cover all generic and specific features of the invention described herein, and all statements of the scope of the invention which, as a matter of language, might be said to fall therebetween.

1. In a personal monitor device, a method comprising the steps of:
measuring a metabolic analyte in a biological fluid of a user, and
correlating the metabolic analyte to one of an amount of body fat of the user and a change in the amount of body fat of the user.

2. The method of claim 1, wherein the step of measuring further comprises the step of measuring a ketone level.

3. The method of claim 1, wherein the step of measuring further comprises the step of measuring a beta-Hydroxybutyrate level.

4. The method of claim 1, wherein the step of measuring further comprises the step of measuring a glycerol level.

5. The method of claim 1, wherein the step of measuring further comprises the step of measuring a free fatty acid level.

6. The method of claim 1, wherein the step of correlating further comprises the step of correlating an amount of body fat lost by the user to an expected change in body weight of the user.

7. The method of claim 1, further comprising the step of calculating a calorie deficit incurred by the user.

8. The method of claim 1, further comprising the step of calculating an amount of calories for the user to consume while maintaining weight loss.

9. The method of claim 8, further comprising the step of displaying a selection of meals having the amount of calories calculated in said step of calculating.

10. The method of claim 8, further comprising the step of displaying one or more instructions to the user.

11. The method of claim 10, further comprising the step of displaying instructions related to testing activity.

12. The method of claim 10, further comprising the step of displaying instructions related to exercise.

13. A device, comprising
measurement means for measuring a metabolic analyte in a biological fluid of a user, and
a processor for correlating the metabolic analyte to a change in an amount of body fat in the user.

14. The device of claim 13, wherein the processor correlates an amount of body fat lost by the user to an expected change in body weight of the user.

15. The device of claim 13, wherein the processor calculates a calorie deficit incurred by the user.

16. The device of claim 13, wherein the measurement means comprises a test element for the metabolic analyte of the user.

17. An apparatus for measuring and correlating a metabolic analyte in a sample to one of weight loss and body fat loss comprising:
a sampling device for receiving a biological fluid from a user, the biological fluid including a metabolic analyte,
a test element for measuring an amount of the metabolic analyte in the biological fluid, and
a processor for calculating the amount of the metabolic analyte in the biological fluid, and for correlating the metabolic analyte to one of an amount of body fat of the user and a change in the amount of body fat of the user.

18. The apparatus of claim 17, further comprising a display for displaying selected data to the user.

19. The apparatus of claim 17, wherein the processor comprises
a calculator for calculating the amount of the metabolic analyte in the biological fluid, and
a correlator for correlating the metabolic analyte to one of an amount of body fat of the user and a change in the amount of body fat of the user.

20. In a personal monitor device, a method comprising the steps of:
measuring a metabolic analyte in a biological fluid of a user, and
correlating the metabolic analyte to a biological parameter of the user.

21. The method of claim 20, wherein the biological parameter comprises one of a change in an amount of body fat in the user, a change in the amount of body fat in the user, a metabolic rate of body fat of the user, weight, caloric consumption, calorie burning rate.
